(19) United States
(12) Patent Application Publication
(10) Pub. No.: US 2008/0262344 A1
(43) Pub. Date: Oct. 23, 2008

(54) RELATIVE VALUE SUMMARY PERFUSION MAP

(76) Inventor: David P. Brummett, Columbia, MO (US)

Correspondence Address: LATHROP & GAGE LC, 2345 GRAND AVENUE, SUITE 2800, KANSAS CITY, MO, 64108, US

(21) Appl. No.: 11/738,900

(22) Filed: Apr. 23, 2007

(51) Int. Cl. A61B 6/00; A61B 5/05

(52) U.S. Cl. 600/426

(57) ABSTRACT

Disclosed is a system and method for diagnosing a perfusion condition in the human brain. The process used includes measuring parameter values at a location of interest in one hemisphere and determining relative differences between those measured values against values measured at a substantially symmetrically opposite location in the other hemisphere. These relative differences are used to generate a composite output which is displayed using different colors. Each color is reflective of a different tissue state, e.g., ischemic core, penumbra, etc. If the substantially symmetrically opposite location is abnormal, and thus, is not a good reference, an alternative reference location which is not substantially symmetrically opposite the location of interest is chosen, and a relative difference map generated. Alternatively, an absolute value map can be generated.

### BACKGROUND OF THE INVENTION

1. Field of the Invention

The invention relates generally to the field of using computed tomography (CT) for perfusion imaging. More specifically, the invention relates to a system and a method for generating map and summary values for the purpose of enabling a user to more easily and quickly review of the CT perfusion data.

2. Description of the Related Art

Stroke is one of the leading causes of death and disability. The condition typically causes a sudden loss of neurological function due to decreased perfusion of the brain with oxygen and glucose. The decrease in perfusion can injure the affected brain cells—a condition referred to in the art as “ischemia.” If the decreased perfusion continues, the cell will be irreversibly damaged and eventually die. This is called infarction or stroke. The group of cells that have been damaged beyond the point of recovery have also been referred to as the ischemic core. If the decrease in perfusion has only occurred for a short period of time and/or if the brain has a good compensatory mechanism, a portion of the injured brain can be salvaged if perfusion is restored. This is called the penumbra. There is another clinical diagnosis called a transient ischemic attack (TIA) where there is a decrease neurologic function which resolves in 24 hours. This is likely reduced perfusion without significant cell injury. Perfusion can be restored naturally by the body or by medical therapy consisting of chemical or mechanical thrombolysis (i.e. clot destruction).

But the decision to implement thrombolysis is not without risk. The unnecessary implementation of thrombolysis where the damaged cells are beyond repair can lead to hemorrhage without the benefit of restoration of blood flow. Therefore, prompt decision correctness is paramount.

To that end, perfusion imaging with CT, magnetic resonance imaging, single photon computed emission tomography (SPECT) and positron emission tomography (PET) have allowed the measurement of perfusion parameters to aid in stroke detection, and quantification. Because of factors such as equipment and technologist availability as well as the speed of imaging acquisition, CT is often the most reasonable modality to evaluate perfusion of the brain.

Perfusion CT is a process where blood flow parameters of the brain are evaluated and measured by observing the change in density or attenuation of imaged structures as iodinated contrast is injected intravenously into the patient.

The parameters measured include Cerebral Blood Flow (CBF), Cerebral Blood Volume (CBV), Time to Peak (TTP), and Mean Transit Time (MTT). The values for these parameters can be provided numerically and in the form of color maps to help aid in diagnosis of stroke and to show the perfusion parameter value for an area of the brain.

During a stroke, the MTT and TTP values increase significantly and the CBF usually decreases. When this occurs, the brain compensates for the decreased CBF by dilating the capillary bed to in an attempt to preserve flow. This causes the CBV to increase or stay near normal. With sustained hypo-perfusion, however, the auto-regulatory mechanisms fail and the CBV decreases enough that tissue is no longer salvageable. In these instances, the patient will likely have permanent injury. It has been proposed that the TIA can have a slight increase in MTT or TTP with normal or near-normal CBF and CBV values. Utilizing these parameters can be a practical problem because there is a tremendous amount of data to process in a very short period of time. The longer the decreased perfusion occurs, the more the brain is damaged. On the other hand, if therapy is incorrectly applied to brain that is not salvageable not only will brain cells not respond to the therapy, but the risk of therapy-induced brain hemorrhage increases.

There are some proposed methods in existence that are designed to further clarify information obtained with a perfusion scan. The proposed methods, however, have limitations.

One proposal (see, e.g., U.S. Pat. No. 6,792,302 issued to Wintermark et al.) relies upon absolute perfusion values for the determination of tissue state. The normal CBV and CBF values are substantially different between gray and white matter of the brain (1, 2). Using a value which is specific for gray or white matter can result in incorrect characterization of tissue type not encompassed by that value. Further, reliance on absolute values also does not completely allow for variations that exist in these values even among normal patients, as there is a range of values that are considered normal. For instance, if the majority of a patient's brain for a particular parameter is just above or at the absolute threshold value for determining normal versus abnormal there may be less confidence in the making the diagnosis of normal versus abnormal than compared to a patient who has the majority of their perfusion values well above that threshold value. Another problem is that the absolute value numbers generated can also vary when different operators of the CT scanner and post-processing equipment are performing the study (3, 4, 5). The absolute perfusion parameter value can also differ depending on the region of interest chosen, particularly for the reference arterial and venous designation, in processing the data (4, 5). Perfusion values can also differ with scanners produced by the different CT manufactures because some of the physics of the equipment and perfusion techniques vary among the vendors. Because of this, the user of the Wintermark technique may not be as accurate in making emergency treatment decisions.

Another proposal discussed in U.S. Patent Application Publication No. 2005/0283070 made by Imielinska et al. uses relative values taken from one location compared to values taken at another location. But Imielinska only incorporates individual perfusion parameters in generating relative difference data and maps for each parameter. Thus the Imielinska maps are all separate and each parameter is displayed relative to specific thresholds for the parameter. Because the parameters must be separately evaluated, the individual using this model has to review more than one map and relate the findings of the individual maps to each other to arrive at a conclusion which incorporates more than one parameter. The different parameters also have particular strengths and weakness in determining tissue state (1). Because the data images are not at all interrelated, the parameters cannot be weighted with respect to their particular strengths or weaknesses when displaying the perfusion data. All of this causes the individual using this data to lose valuable time when evaluating a patient.

### SUMMARY

The present invention is defined by the claims below. Disclosed embodiments of the disclosed present invention include a process for diagnosing a perfusion condition in the human brain. One embodiment of the disclosed process includes selecting an anatomical location of interest in the first hemisphere of the brain. Then, a substantially symmetrically opposite location is located in the other hemisphere. The process then measures a first set of values for a given number of parameters at the location of interest. The process also measures a second set of values for the same parameters at said substantially symmetrically opposite location in the other hemisphere. A determination is made of the relative differences between said first and second sets of values. Given this determination, the relative differences are used to generate a composite output which reflects the condition at the location of interest.

The composite output, in one embodiment, is displayed as an image in some fashion, e.g., on a computer display device. A plurality of colors can be used to differentiate between different tissue states in the image. This displayed image can be adapted such that it comprises a perfusion map.

In another embodiment, the step of using relative difference values to generate the composite includes a step of classifying the damaged tissues, e.g., ischemia, decreased perfusion without substantial cell injury, penumbra, infarct) into different perfusion states utilizing threshold values applied to each of the parameters measured (e.g., one or more of MTT, CBV, CCF, TTP).

In another embodiment, a query is made. In this query step, it is determined whether the tissues at both the location of interest and the substantially symmetrically opposite location are abnormal. If so, then the process is directed to an alternative method of diagnosing the perfusion condition. But this will only be executed if both of the location of interest and the substantially symmetrically opposite location are abnormal.

One possible alternative method includes selecting an alternative reference location which is not substantially symmetrically opposite the location of interest. This alternative reference location will be at a location where tissues are in normal condition. It may be in either hemisphere. Once this alternative reference location is selected, parameters are measured to determine an alternative set of values at the alternative reference location, and relative differences are determined between the first set of values and the alternative set of values. These alternative relative differences are then used to generate the composite output which is reflective of the condition of tissue at the location of interest.

Another possible alternative method for use when the tissue substantially symmetrically opposite said location of interest is abnormal is to generate a summary map using absolute values.

### BRIEF DESCRIPTION OF THE SEVERAL VIEWS OF THE DRAWINGS

Illustrative embodiments of the present invention are described in detail below with reference to the attached drawing figures, which are incorporated by reference herein and wherein:

FIG. 1 Illustrates the CT table, CT scanner, and position of the patient before the table positions the patient's head into the CT scanner gantry for imaging.

FIG. 2 Illustrates the CT scanner, patient with head in the gantry (circle in the middle of the scanner), computer utilized to control the scanner and collect initial data, display unit, independent processing equipment and data storage device.

FIG. 3 Flow diagram that describes the sequences of events utilized in this model. It shows the initial imaging performed and how data is further processed.

FIG. 4 Illustrates a non-contrast head CT with orientation of head in the transverse axial plane. This is the plane depicted to the operator as the scanner acquires data and how it is initially reviewed and processed by the operator. The basal ganglia are Para-midline structures depicted to better show patient orientation. This is also a common place where perfusion images are obtained.

FIG. 5 Illustrates a contrast enhanced CT. An example of a reference artery and vein is depicted.

FIG. 6. Illustrates data that can be obtained as a perfusion scan. The region of the brain that is abnormal is depicted by colors. The different colors—represented by distinctive cross-hatching—correspond to different perfusion values of the tissue in the abnormal region.

FIG. 7 Illustrates the Symmetry/Quality assurance map depicting the abnormal region and depicting the normal reference region in the opposite hemisphere.

FIG. 8 Illustrates the Primary Perfusion Summary Map. The cross hatching is used in the figure to reflect distinct coloring. The distinct coloring in different map regions is used to indicated regions in which perfusion values are abnormal. The different colors correspond to tissue classified in different perfusion states as derived using the model outlined in this invention. The reference tissue is chose automatically based on the distance from midline.

FIG. 9 Illustrates an alternative Summary Map where the operator of the exam selects the reference anatomy.

FIG. 10 Illustrates the alternative Summary Map utilizing absolute values.

### DETAILED DESCRIPTION

The disclosed invention relates to a system and a method for generating map and summary values that will simply review of the CT perfusion data. The map created will primarily utilize relative difference in perfusion among different brain regions and will be displayed as a composite image displaying a plurality of parameters at once. The diagnosing and treating physicians can then more quickly and accurately evaluate the patient's condition to determine whether to initiate or withhold thrombolytic therapy. The map can also provide general prognostic information. Because the process enables quantifying brain ischemia in a summary fashion, deciding which therapy option to use on the patient is accomplished more easily and faster. Further, the process is able to provide basic prognostic information regarding the degree of current brain injury and amount of injury that can exist if adequate perfusion is not restored with more accuracy than can be accomplished with the prior art methods. Further, the process enables images to be processed and displayed so that they have more clinical relevance than is possible with current techniques. The method utilizes data that is acquired by established perfusion CT techniques. One skilled in the art will be aware of established perfusion techniques can measure the MTT, CBV, CBF, and TTP of the brain parenchyma.

The primary embodiment of the method involves measuring the relative difference or percentage difference of perfusion values between the two hemispheres of the brain, comparing that difference to predetermined thresholds, and classifying the brain parenchyma perfusion state utilizing a plurality of parameters simultaneously. The tissue designation is then displayed as color map or image which classifies tissue with reduced perfusion into one of three tissue states.

To compare the brain from side-to-side an axis of symmetry must first be assigned. Once this midline axis is established, one side of the brain can be evaluated relative to the other side as the tissue is measured relative to location from the midline structure. One skilled in the art will know how to incorporate a midline brain designation into generation of perfusion values. The brain is symmetric anatomically side-to-side, provided the patient has not had a rare congenital anomaly, or previous injury has rendered the tissue non-symmetric. Provided the subject brain is free of this sort of anomaly, injury, or other irregularity, evaluating tissue that equidistant from the established midline on either side of the brain to each other, is evaluating tissue that is symmetric to each other, and should render substantially equal readings.

One side of the brain can therefore be the reference for the other side for a particular region of interest or anatomy. The percentage reduction or increase of a perfusion values comparing one side of the brain relative to the other can then be calculated from the quantified perfusion parameters obtained by standard perfusion imaging methods.

In the preferred embodiment, the present process model will utilize more than one parameter when quantifying and displaying brain tissue perfusion state. In the preferred embodiment, the MTT and CBF are the selected parameters to evaluate for ischemia. These parameters will be utilized for detecting significantly reduced perfusion and will be evaluated comparing the values from one side of the brain relative to the other. The relative difference in these values will be compared against established threshold values. Deviations from these threshold values are then communicated to the user so that he or she may determine whether or not substantially reduced perfusion has occurred. Additionally in the preferred embodiment the CBV is then compared from one side of the brain relative to the other to establish whether or not there is a relative difference which could indicate that the tissue with reduced perfusion is infracted or less likely to recover if adequate blood flow or perfusion is restored, as opposed to penumbra. The relative difference that exists in CBV between hemispheres will be also compared to threshold values in determining whether or not infarction has occurred. In the preferred embodiment, the results of the comparison of relative values for MTT, CBF, and CBV to the threshold values are then displayed as a composite image which depicts the tissue state. The perfusion map image therefore accounts for multiple parameters for a particular slice of tissue or imaging level. Further, the tissue with reduced perfusion may be highlighted and assigned a color value indicating its perfusion state. The colors will classify reduced perfusion tissue as being in one of three states that is represented by one of three colors. It is also possible, however, that fewer (e.g., two) or more (e.g., four or more) states could be identified and then displayed depending on the objectives of the user. Thus, it should be realized that the scope of the disclosed processes should not be necessarily so limited.

It is possible that an anomaly such that those discussed above exists in the subject brain which does not permit the side-to-side comparison to be ideal because the anomaly has caused the side of the brain that is not of acute clinical interest for a stroke to be abnormal and therefore, not useable as a reference for normal tissue. When this occurs, the hemispherical comparison may not provide reliable information. Thus, in order to address this situation, two alternative perfusion summary maps may also be constructed and displayed for the cases when both hemispheres have abnormal perfusion values for a particular region of anatomy. Determination of symmetry with respect to damage in both hemispheres for a region of interest can often be made on inspection of precontrast CT images or gross inspection of perfusion images; however, a symmetry map can also be created for review to better depict symmetry as well as overall exam quality. A symmetry map will be created where the values obtained for a particular perfusion parameter are compared to quantifiable known standards to determine if a specific anatomic region has an abnormal value for that parameter in both hemispheres. This information can enable determination of symmetry for a particular region of interest with respect to using one side as a reference for normal tissue, as well as evaluate overall exam quality. One alternative map option allows region of interest to be done manually by the person performing the exam. This will permit the region of clinical interest to be compared to another region of more normal appearing tissue as seen on the symmetry map or individual perfusion maps. The tissue will be compared as a relative value of one brain region to the other. This can be done using a variety of perfusion parameters. MTT for evaluation of ischemia and CBV for evaluation of ischemic core is preferred in this embodiment. Utilizing these two parameters, reduced perfusion tissue can be classified in one of the three tissue states listed above.

The other alternative map will use absolute values to classify the brain tissue. The preferred method would utilize MTT to evaluate for ischemia and CBV to evaluate for ischemic core. The tissue with reduced perfusion would then be classified in one of three tissue states, as it was with the other two perfusion summary map options.

The processes and systems of the present invention are not necessarily tied to any particular environment. For example, the disclosed processes and systems can be applied to other imaging modalities that can produce perfusion parameters. For example, besides CT, the processes disclosed herein would also be useable with MRI, PET, SPECT, and possibly other imaging techniques. Therefore, the scope of the present invention should not be limited to any particular imaging process unless specifically claimed. In the preferred embodiment, however, CT has been selected.

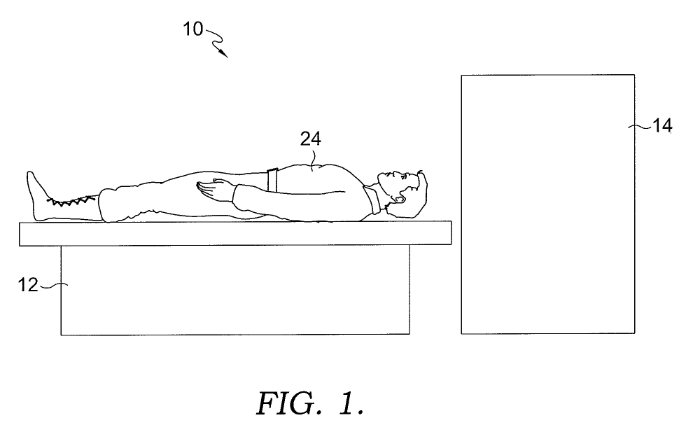

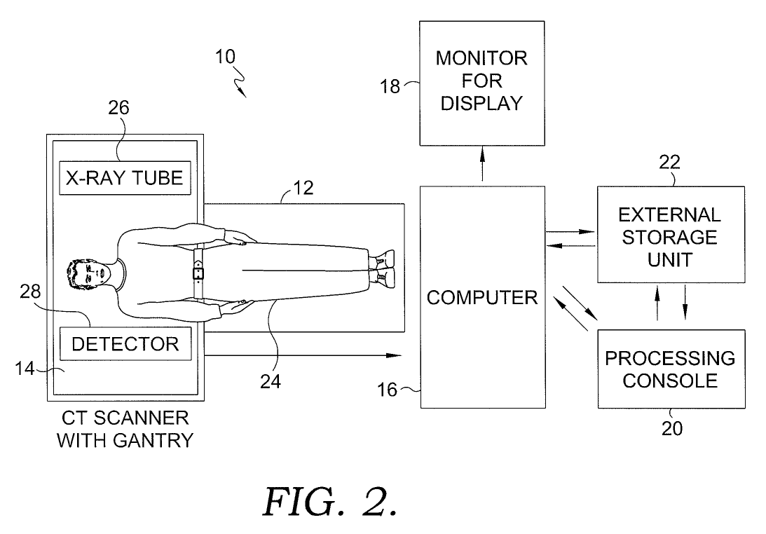

A typical CT imaging system is disclosed in FIGS. 1 and 2, which is one environment in which the process might be used. Such systems are well known in the art and used conventionally to acquire images of a patient's brain. Referring to the drawings, the basic elements of a CT imaging system 10 are a CT table 12, a gantry 14, an operating consol and computer 16 which is often included in the same unit, a display monitor 18, a workstation to process the data 20, and a data storage device 22. A patient 24 is placed on table 12 and positioned within CT gantry 14 by motorized movement of the table in a known manner. The specific functions of the table and CT unit are usually performed at operating console 20 or directly at computer 16. X-ray beams are produced in the gantry by an x-ray tube 26. The x-ray beams generated pass through patient 24, are then attenuated by the tissue of the patient and then reach detector elements in a detector device 28 on the opposite side of gantry 14. Table 12 moves during image acquisitions so that different portions or slices of patient anatomy along the z-axis (axis with head to toe orientation) are covered. The data from detector 28 is then received by computer 16 and then processed between analog and digital data states so that the information can be shown on display 18. Conventional processes are used to transform the detected information into a form enabling that information to be constructed into a useable image in real time, and also enable storage of this image information such that it can be reviewed. Real-time and/or reviewable images can then be displayed on display unit 18.

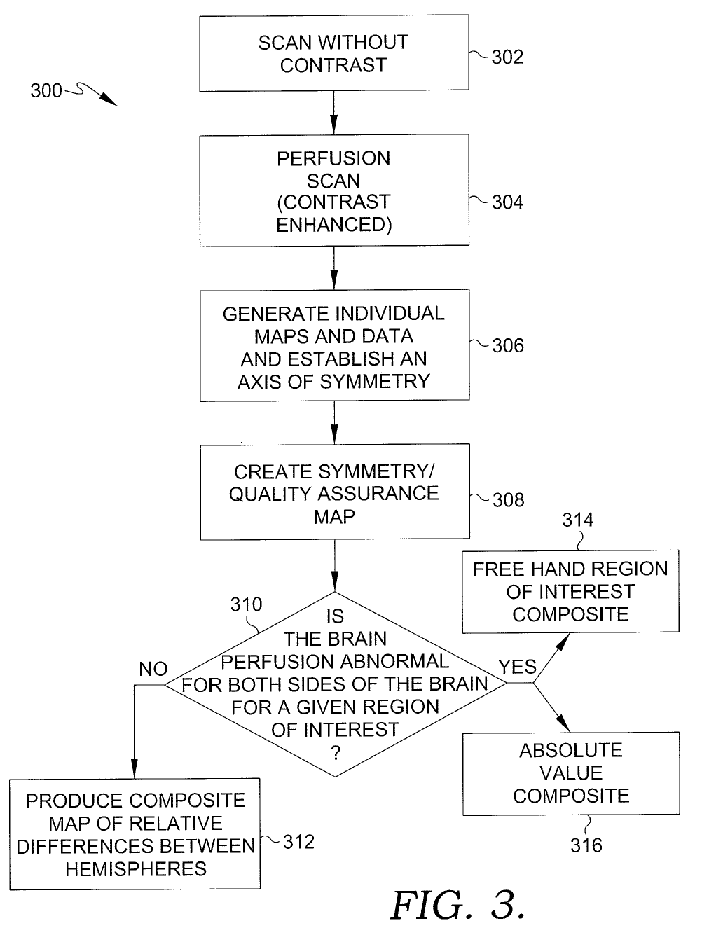

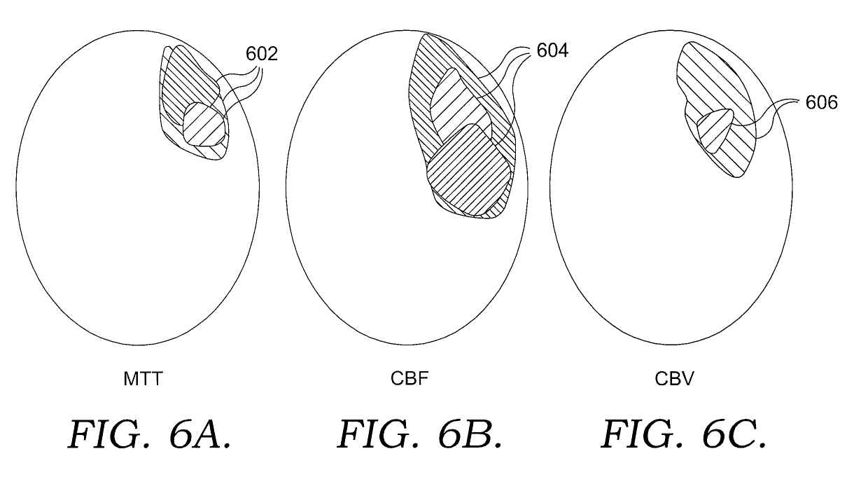

The flow diagram of FIG. 3 shows an embodiment 300 for the present process. Using established techniques (deconvolution or absolute slope model) and existing software, flow parameters can be calculated and display as color images for each parameter. The parameters of interest that can be obtained include MTT, TTP, CBV, CBF. The colors of the map display for each parameter can be made to correspond to various perfusion values. MTT, CBF, and CBV are shown in FIGS. 6A-C, respectively. As can be seen in the figures, different perfusion parameters values are represented by different colors (seen as different cross-hatch marks here) for each parameter. The varieties of tissue conditions in areas 602, 604, and 606 are distinguished by some method. Here, they are identified by different colors.

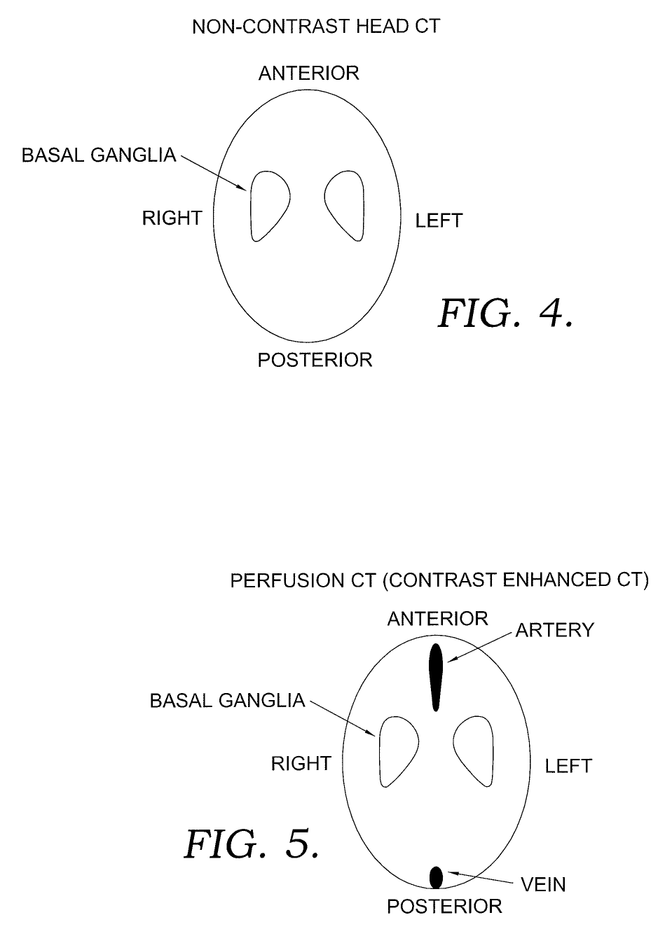

In a step 302, A non-contrast CT exam of the brain is performed. This test is performed prior to the introduction of any contrast material. A typical result for such a test is shown in FIG. 4. The purpose for doing this prior to taking a perfusion CT (see, e.g., FIG. 5) is to evaluate the condition of the subject brain for pathology such as a prominent area of hemorrhage, or other irregularities that can be seen without contrast enhancement and which can sometimes negate the need for a perfusion CT.

In a step 304, once it is determined that obtaining a perfusion CT is desirable, a contrast enhanced perfusion scan is taken. As will be known to those skilled in the art, the patient is normally administered a contrast material via a power injector device. The contrast material changes the attenuation or density value of arteries, veins, and brain parenchyma as it moves through these structures. Once the contrast material has been administered, the user/technician will begin the scan in a manner known to those skilled in the art. Scanning at a particular level in the brain begins before contrast is detected, and continues as contrast goes from the arteries in the brain parenchyma and then into the veins.

In a next step 306, the enhanced perfusion scan creates an analog signal that is received by computer 16, converted into a useable digital data files, and generated into useable individual maps and other data that are reconstructable using the combined system comprising computer 16, processing console 20, and storage unit 22 in a manner known to those skilled in the art. Once this has been accomplished, the present method takes the data generated and constructs individual MTT, TTP, CBV, and CBF maps. Each of these maps are then depict the general values for one parameter as a single image per slice of anatomy covered. Therefore, there can be multiple maps for each slice covered with each map conveying information about a single perfusion parameter.

Also part of step 306, is the generation of the subject brain axis of symmetry. One skilled in the art will recognize that the axis of symmetry is established in obtaining the routine perfusion data and maps. If it is not already designated, an axis of symmetry can be established by determining midline off of the generally oval shaped brain, when viewed in a transverse axial projection, or utilizing a midline brain structure such as the septum pellucidum, third ventricle, or falx cerebri. This axis can then be used to find laterally equivalent symmetrical position on each side of midline.

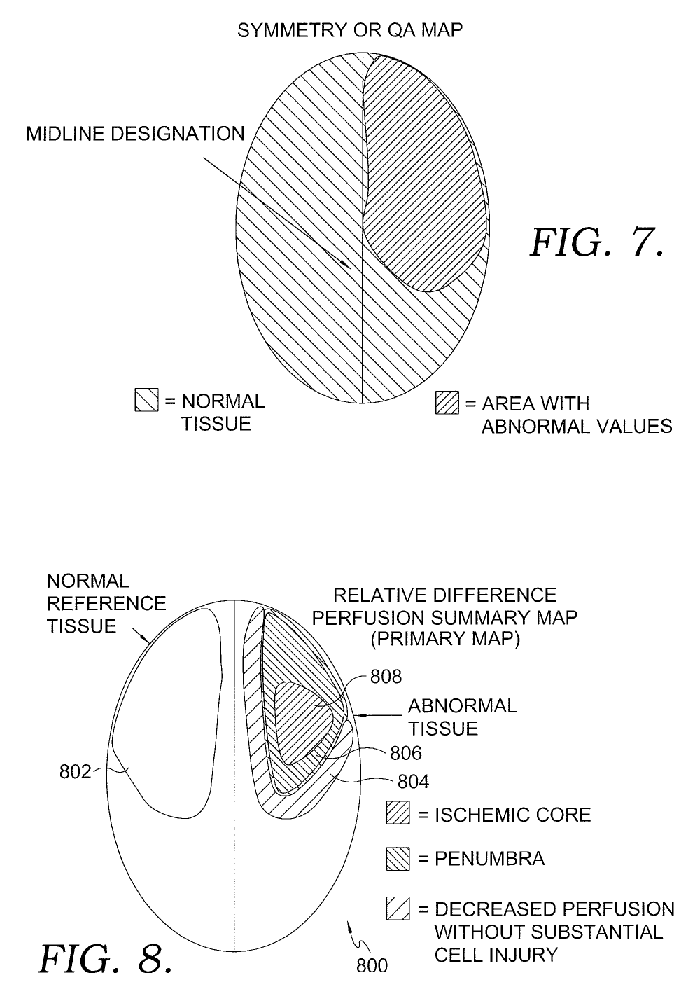

Next, a step 308 involves the creation of a symmetry/quality assurance map (see, e.g., FIG. 7) using the referenced axis of symmetry. This map is generated by reviewing the data in a particular map relative to known normal values to determine if a particular area of the brain in both hemispheres has abnormal data that could compromise performance of side-to-side comparison. As stated above, the symmetry/quality assurance map will also quantify the overall validity of the study data. Objective evaluation of overall exam quality is important because if the exam is not performed technically correct manner, with respect to image acquisition or post-processing, the perfusion parameter values generated can be abnormal for all parts of the brain imaged and therefore are of limited value. Although any parameter could potentially be used for such a map, MTT is preferred because it does not differ significantly between white and gray matter and because it is very sensitive for detection of ischemia.

Once the MTT (or other parameter) symmetry/quality assurance map has been generated, it is used to make a determination in a query step 310 as to whether sufficient symmetry exists which would enable accurate cross axis comparison. In one disclosed embodiment, this is done by comparing the values produced on a particular image to an MTT value that is established as being more normal. But the particular value designated can vary depending on imaging equipment, imaging protocol, and processing equipment utilized. Because of this, the value used to determine quality assurance must be established with consideration to particular imaging parameters utilized. Step 310 could be accomplished entirely through automation, or could be a subjective determination made by an exam operator or interpreter. Regardless, this step is advantageous in that it properly directs the process so that the most effective mapping is executed.

If sufficient symmetry exists (a “no” answer to query step 310), and thus, the data between hemispheres is considered acceptable the process moves on to a step 312 in which a composite relative difference summary map is created, in which the relative differences are taken across the axis of symmetry at exactly opposite locations, which in the normal healthy brain should have equivalent values. This makes the cross-axis equivalent locations in the brain a superior reference from which to determine the existence of abnormal values which would tend to indicate ischemia.

The primary perfusion summary map and data values produced in step 312 in the process of the preferred embodiment are based on the relative differences in MTT, CBF, and CBV between the normal and abnormal tissue. It is also, however, possible that other parameters or combinations of parameters can be used instead. For example, TTP could potentially be substituted for MTT or a single ischemia parameter of MTT, CBF, or TTP could be used instead of combination of MTT and CBF, although MTT and CBF are shown in the embodiment discussed thus far. As stated above, the brain is a symmetric structure from side-to-side so the abnormal side can be examined using the opposite side as a reference for normal tissue parameters. The present method calculates and displays the percentage difference in MTT, CBF, and CBV between the hemispheres and denoting critical percentage values based on predetermined thresholds.

For example, an MTT value in the damaged location which is greater than 130% of the value in the opposite reference undamaged hemisphere, but less than 145% in the opposite undamaged hemisphere will indicate reduced perfusion without significant cell injury. Also, either of: (i) a MTT value in the damaged location which is greater than 145% of the MTT reference value taken from the opposite locations in the healthy opposite hemisphere, or (ii) a CBF value that is less than 60% of the value taken from the opposite location in the reference opposite hemisphere would indicate ischemia. Further, a measured CBV value that is less than 40% of the value read from the referenced opposite hemisphere will indicate ischemic core. Thus, because the above-noted differentials have been identified as determiners of the particular state of the damaged portion, they are very useful in making a prognosis.

Recognizing this, and using known computing techniques, FIG. 2 system has been adapted to separately identify different regions of the brain based on status according to the differentials measured. Thus, if the measured values in regions where the brain tissue has: (i) MTT values greater than 130% of the referenced location, but less than 145% of the referenced healthy location, (ii) CBF values that are greater than 60% of the referenced locations, and (iii) CBV values that are greater than 40% of the corresponding values in the reference hemisphere, that region will be designated by some identifying technique as reduced perfusion without significant injury. If the measured values in a region of tissue have MTT values greater than 145% of reference, or CBF values less than 60% of reference, and CBV values greater than 40% of the corresponding locations in the opposite/reference hemisphere, that region will be identified as penumbra. If the measured values in a region of tissue have MTT greater than 145% of reference, or CBF less than 60% of reference, and CBV less than 40% of the reference location in the opposite hemisphere, that region will be labeled as ischemic core, and thus, less likely salvageable.

It should be noted that although example discussed above discloses particular threshold ranges and values that the disclosed processes should are not be limited to any particular threshold value that might serve as a trigger. Generally speaking, it should be recognized that tissue with a measured MTT that is slightly prolonged with normal CBF and CBV values relative to the reference (taken from a more normal opposite hemisphere) would likely be designated as having reduced perfusion without significant detectable cell injury. An increased MTT, decreased CBF and CBV that is increased, normal or has a relatively a small decrease will indicate penumbra. A prolonged MTT with significant relative decrease in CBF and CBV will indicate ischemic core.

Regardless, once the designations of tissue state have been determined by the process, they, in one embodiment, will then be displayed in a color map as a composite image that stratifies the brain with reduced perfusion into one of these three categories. FIG. 8 shows such a display 800 in which a normal reference tissue area 802 is shown on one side of the axis, and an area of decreased perfusion without substantial cell injury 804, a penumbra area 806, and an ischemic core area 808 are all displayed. In the disclosed embodiment, each area is identified by color coding. But other possibilities exist for identifying and distinguishing relative tissue status. For example, shading, labeling, or other means to distinguish areas could be used instead. It is also possible that different means, such as tables, could be used to distinguish between regions which satisfy different criteria in terms of differentials from the reference values taken from the healthy side of the brain.

If, at step 310, the process (through automation or human intervention) determines that there is a significant defect in both hemispheres pertaining to the region of clinical interest, the answer at step 310 will be “yes,” and one of two alternative summary maps can be utilized. These alternatives are reflected in embodiment 300 as a split into either of: (i) a step 314 where the differentials are taken relative to a free hand selected region of interest, or (ii) a step 316 in which an absolute value composite is produced. The determination of which step the process is directed to can be managed in any number of ways. With the disclosed embodiment, a human determination is made. More specifically, the operator reviews the data and makes a determination of which of the two alternative steps 314 or 316 are preferred given the data collected regarding the subject brain being examined.

Step 314, the first alternative process involves the creation of an alternative map that can be utilized includes a relative difference summary map with regions of interested chosen by the exam interpreter or operator. In this process, the user/operator selects an alternate reference location because it has been determined in earlier step 310 that the necessary symmetry required for the direct cross-hemispherical referencing technique does not exist. Thus, as a fall back position here, the operator can chose anatomy in the same hemisphere or opposite hemisphere that appears more normal on the symmetry map or individual perfusion parameter maps.

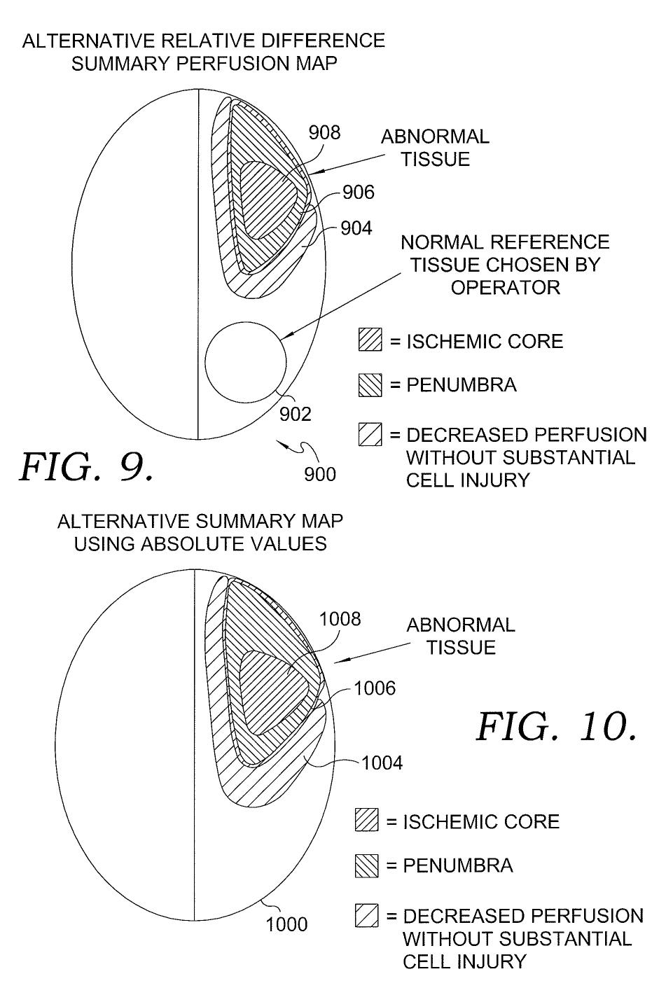

A map 900 which might exemplify this is shown in FIG. 9. Referring to the figure, it can be seen that a normal reference location 902 has been selected by the operator. In this example, the location is in this same hemisphere. This is done by inspection of the symmetry or individual perfusion maps to determine which tissue is normal and can therefore be used for reference and for more abnormal appearing tissue on these same maps. The operator will then select this region of interest, preferably in tissue with a similar composition of gray or white matter relative to the tissue in question for acute ischemia. By selecting tissue with similar gray and white compostition, the relative comparison is more accurate as these parameters can be different between gray and white matter for CBV (and CBF) as stated above.

Alternatively, the operator might select a reference area in the opposing hemisphere. This is again complished by selecting more normal appearing tissue on the other maps and trying to encorporate similar gray/white matter compostion to the region suspected of having a stroke. In the preferred embodiment, MTT and CBV have been selected as the primary parameters utilized. MTT is chosen because it does not vary significantly between white and gray matter. CBV is chosen because it is more sensitive to ischemic core. In other words, CBV more accurately designates tissue as being ischemic core when it exists compared to MTT, CBF, and TTP. If MTT is slightly prolonged and CBV is normal, this is a strong indication of reduced perfusion but likely without significant cell injury. Thus, the process would then designate these areas (via color coding or some other identifying means) as decreased perfusion without significant cell injury as shown in a region 904. But if MTT is more substantially prolonged and CBV is near normal or only slightly reduced, the tissue is designated as penumbra as shown in a region 906. If MTT is substantially prolonged and CBV is significantly reduced, the tissue is designated ischemic core as shown in a region 908.

Referring to FIG. 10, a second possible alternative map 1000 utilizes absolute values to determine tissue state. In this alternative step, the absolute values generated from the perfusion scan will be compared against predetermined thresholds for the absolute value. If the absolute value of MTT is slightly prolonged and the absolute value of CBV near normal, the tissue is designated by color coding or other means of identification as having reduce perfusion without significant injury, as shown in a region 1004. If the absolute value of MTT is more significantly prolonged and the CBV is near normal or slightly reduced, tissue will be color coded (or otherwise identified) as penumbra, as shown in a region 1006. If the absolute value of MTT is significantly prolonged and CBV significantly low, tissue is designated as ischemic core, and the process may identify it by coloring a region 1008 or other distinguishing means.

Many different arrangements of the various components depicted, as well as components not shown, are possible without departing from the spirit and scope of the present invention. Embodiments of the present invention have been described with the intent to be illustrative rather than restrictive. Alternative embodiments will become apparent to those skilled in the art that do not depart from its scope. A skilled artisan may develop alternative means of implementing the aforementioned improvements without departing from the scope of the present invention.

It will be understood that certain features and subcombinations are of utility and may be employed without reference to other features and subcombinations and are contemplated within the scope of the claims. Not all steps listed in the various figures need be carried out in the specific order described.

The invention claimed is:

1. A process for diagnosing a perfusion condition in the brain of a patient, comprising:
selecting an anatomical location of interest in a first hemisphere of a brain;
identifying a substantially symmetrically opposite location in an other hemisphere of said brain;
measuring a first set of values for plurality of parameters at said location of interest;
measuring a second set of values for said plurality of parameters at said substantially symmetrically opposite location in said other hemisphere;
determining relative differences between said first and second sets of values; and
using said relative differences to generate a composite output which is reflective of said condition at said location of interest.

2. The process of claim 1, comprising:
providing a device to display said output as an image;
using a plurality of colors to differentiate between different tissue states in said image.

3. The process of claim 1, comprising:
adapting said image to be a perfusion map.

4. The process of claim 1 wherein said process of using said relative differences to generate a composite output step comprises:
classifying tissues into different perfusion states utilizing threshold values applied to each of said plurality of parameters.

5. The process of claim 1, comprising:
determining whether tissues at both of said location of interest and said substantially symmetrically opposite locations are abnormal; and
directing the process to an alternative method of diagnosing said perfusion condition if both of said location of interest and said substantially symmetrically opposite location include tissues which are abnormal.

6. The method of claim 5 wherein said alternative method comprises:
selecting an alternative reference location which is not substantially symmetrically opposite said location of interest, said alternative reference location including tissues in a normal condition in one of said first and other hemispheres;
measuring said plurality of parameter to determine an alternative set of values at said alternative reference location;
determining alternative relative differences between said first set of values and said alternative set of values; and
using said alternative relative differences to generate a composite output which is reflective of said condition at said location of interest.

7. The method of claim 5 comprising:
generating an alternative summary map using absolute values.

8. The method of claim 1 comprising:
providing the user with an ability to view output values relating to each of said parameters individually rather than in composite form.

9. The method of claim 1 comprising:
including MTT in said plurality of parameters for the purpose of determining the existence of ischemia; and
including CBV in said plurality of parameters for the purpose of determining the existence of infarct.

10. The method of claim 9 comprising:
including CBF in said plurality of parameters for the purpose of determining the existence of ischemia along with said MTT.

11. The method of claim 1 comprising:
including CBF in said plurality of parameters for the purpose of determining the existence of ischemia; and
including CBV in said plurality of parameters for the purpose of determining the existence of infarct.

12. The method of claim 1 comprising:
including TTP in said plurality of parameters for the purpose of determining the existence of ischemia; and
including CBV in said plurality of parameters for the purpose of determining the existence of infarct.

13. The method of claim 12 comprising:
including CBF in said plurality of parameters for the purpose of determining the existence of ischemia along with said TTP.
